(19) United States
(12) Patent Application Publication
(10) Pub. No.: US 2008/0093210 A1
(43) Pub. Date: Apr. 24, 2008

(54) Consumer virus eradicator

(76) Inventor: John Lee Edwards, Sarasota, FL (US)

Correspondence Address: Jackson Walker L.L.P.;c/o Mark A. Tidwell, Suite 1900, 1401 McKinney Street, Houston, TX, 77010, US

(21) Appl. No.: 11/642,292

(22) Filed: Dec. 20, 2006

Related U.S. Application Data
(60) Provisional application No. 60/751,547, filed on Dec. 20, 2005.
(60) Provisional application No. 60/751,546, filed on Dec. 20, 2005.

(51) Int. Cl. B01D 53/00

(52) U.S. Cl. 204/157.3; 422/186.3

(57) ABSTRACT

Real time purification of contaminated common air is an unsolved consumer health problem inside residences and the workplace, and outside air pollution by industry and transportation. Currently popular consumer grade electrostatic air filters accumulate suspended contaminants onto accumulation plates which require subsequent disposal or cleaning of the highly concentrated contaminates, a maintenance requirement that creates new jeopardy to consumer health. Disclosed are a method and apparatus with several embodiments that structurally eradicate suspended particulates after they have been electrostatically migrated to a hollow, activated dismantling rod. The dismantling rod is typically composed of a durable, UV transmitting material, such as tetrafluoroethylene, or a current carrying metal mesh coated with tetrafluoroethylene, and further over-coated with active photocatalytic material that is energized to create on its surface powerful dismantling agents, including hydroxyl radicals, from natural ambient air elements. Microorganisms, smoke particles, industrial pollutants, odor molecules, and allergens are structurally dissociated into harmless protein fragments and natural molecules when they encounter the dismantling grid purifying agents. The decontamination phenomenon is amplified by imparting, or enhancing, either a negative or positive electrostatic charge to the suspended particles, as they enter the device, and using that to migrate the particles to the activated dismantling rod containing an internal, oppositely charged attraction screen. The method can be used to eradicate particles, including lethal pathogens, that may have a naturally occurring negative, positive, or electroneutral charge. Both contaminants and suspended water molecules are drawn to the dismantling rod in this manner, providing an enhanced supply of raw material from which the decontaminating agents are created. Any trace amounts of ozone molecules produced during the process are also attracted and dissociated back to oxygen within the device. The permanent photocatalytic coating does not require renewal or cleaning, and is designed to use UV energy in a bandwidth that also physically inactivates the replication defenses of passing microorganisms until they can be dismantled, whether in the first pass on during a subsequent air exchange pass. None of the powerful dismantling agents escape the device because of their short half lives; once their job is done all return to the natural elements from which they were made, creating no environmental harm. The method and apparatus are self cleaning and maintenance free.

This application claims priority from provisional filing No. 60/751,547 “Airborne Virus Eradicator” filed Dec. 20, 2005, and provisional filing 60/751,546 “Consumer Virus Eradicator” filed Dec. 20, 2005.

### BACKGROUND OF THE INVENTION

This invention relates broadly to air cleaning devices that remove airborne contaminants of many types, including, viruses, bacteria, mycotoxin, fungi, spores, allergen particles, odiferous molecules, Volatile Organic Compounds, and other Toxic Industrial Chemicals. More specifically, the invention relates to a device that utilizes electromagnetic energy to generate hydroxyl radicals on the surface of semiconductor material, thereby facilitating the removal of airborne contaminants.

There is a critical need to improve the air quality of inhabited areas by eliminating suspended contaminants. Sale of consumer air purifiers have risen dramatically worldwide since Sep. 11, 2001, because of civilian concern for epidemic pathogens (Avian Virus, SARS, Influenza), and their bio-engineered military grade counterparts (Plague, Smallpox).

Currently popular residential air purifiers electrostatically attract contaminant particles to an accumulation plate that has a negative electrical charge. The process of creating ions with an electrostatic field naturally creates a small air movement. Marketers have seized on this silent air movement feature to promote a quiet, but marginally effective product. Periodically, the accumulation plates become covered with highly concentrated contamination and must be discarded or cleaned, presenting a new consumer health risk. Despite their popularity and claims for low energy consumption, such units aren't particularly effective air purifiers according to published reports from consumer product testing laboratories.

Most suspended contaminants have a natural electrostatic charge present on them. Large macromolecules generally have a natural positive charge. For example, tobacco smoke particles, odors and many allergens tend to be positively charged, which helps keep these large particles suspended in air. Small microorganism particles typically have a negative charge on them. Bacteria and their fragments are negatively charged due to their cell wall chemistry. Gram negative bacteria contain negatively charged —COO* groups associated with proteins and lipopolysaccharides in their cell walls. Gram-negative bacterial endotoxins (pyrogens) are also negatively charged as are viruses and most colloids.¹ The ability to place or alter an electrostatic charge on a suspended particle is directly related to its total surface area and its total mass. It is more difficult to alter or increase the natural electrostatic charge on a small contaminant particle than on a large particle.
¹ Argonide Corp Nanocerm under Table 3 Filtration Mechanisms.

Consumer air purifiers tend to use the natural positive charge that large particles and molecules possess to attract them to a negatively charged collector plate. This negative collector generally minimizes the production of ozone by the devices, the concentration of which is regulated by governmental agencies because of its oxidizing toxicity to human lung material as well as to pathogens. Such consumer devices are inherently less effective at removing smaller bacteria and viruses, which are inherently negatively charged.

Electrostatic technology itself is fundamentally sound, and a mainstay of industrial effluent abatement. Very large, expensive, constantly maintained industrial systems are in use worldwide at industrial plants. These industrial systems generally use positively charged collector plates. They first increase the electronegativity of the generally large effluent particles by strong field ionization, then migrate the particles to large, parallel, positively charged collector plates. Byproduct ozone generated in the process is exhausted to the environment.

### BRIEF SUMMARY OF THE INVENTION

The present invention uses hydroxyl radicals from a photocatalytic reaction to molecularly dismantle pathogens and other contaminants suspended in a breathable airstream. The invention uses electromagnetic energy from conventional plasma discharge lamps in the ultraviolet 200 nm to 400 nm wavelength range to generate hydroxyl radicals on the surface of certain semiconductor materials. Hydroxyl radicals are very potent one-electron oxidants that steal hydrogen molecules from passing organic material, as they seek to replace their missing covalent electron, leaving decayed carbon ions in the organic matter.

There are several types of nanocrystal based semiconductor formulations that will express hydroxyl radicals on their surface via the dissociation of H₂O ambient humidity water molecules into OH hydroxyl radicals. The water vapor is consumed, but cycled back to natural elements including water vapor. The end result of this photochemical conversion and air purification is eventually water and decayed carbon based material so that the cleaning is truly a bio-friendly process.

The presence of hydroxyl radicals on nanocrystal semiconductor surfaces can be confirmed by the use of a clear, colorless chromophore, Nitroblue Tetrazolium (“NBT”). NBT is a chemical substrate used in medical research to measure the bacterial killing ability of human neutrophils, primarily whether they are producing phagocytic singlet oxygen ¹O₂, normally generated in such healthy white blood cells of the immune system. Singlet oxygen is a powerful, endogenous oxidant, second to hydroxyl radicals in its thermodynamic oxidation potential. Singlet oxygen and hydroxyl radicals will both reduce colorless NBT solution to an indigo dye-based chloride salt precipitate, thereafter displaying a deep blue visible color if either has been present to cause the reaction.

The invention eradicates suspended contaminants in the air of enclosures that may still be occupied by people, plants and livestock and to the reduction of industrial airborne pollutants currently being exported to the environment. More particularly, this invention separates suspended contaminants by migrating them to an activated surface, then structurally dissociates them to the molecular level in a device that is self-cleaning and causes no environmental harm. All of the powerful dismantling agents used in the eradication process return to the natural elements from which they were made once their job is done, never escaping the device and causing no environmental harm.

Accordingly, it is a primary object of the present invention to provide a new and improved method and apparatus for destroying contaminants thereby.

It is a further object of this invention to permit operation of the method and apparatus while people, pets, plants, or livestock may be present in the air being treated.

It is a further object of this invention to improve breathable air and in so doing, use no ingredients and leave no residue that could be harmful to the environment.

It is a further object of this invention to separate and migrate suspended contaminants to an eradication site that is self-cleaning.

It is a further object of this invention to eradicate all migrated contaminants by structural dissociation of their cellular or chemical makeup, converting them to non-threatening forms, down to the level of natural environmental molecules.

It is a further object of this invention to amplify the dismantling effect itself by migrating ambient water molecules to a dismantling site to enhance the proximate raw material needed to enhance total photocatalytic activity.

It is a further object of this invention to provide several apparata embodiments for carrying out methods that achieve the foregoing objects and which are relatively simple in construction and effective in operation.

It is a further object of this invention to provide such methods and apparata that can easily be used in either a portable configuration by one person, or permanently incorporated into the conventional air handling systems of industrial and residential buildings.

It is a further object of this invention to provide such methods and apparata that can be permanently incorporated into the air handling systems of transportation vehicles such as aircraft, trains, and busses.

It is a further object of this invention to provide a method and apparatus that can be permanently incorporated into the air waste stream of industrial facilities and health care facilities in order to minimize toxic industrial chemical pollution and hazardous pathogen emissions to the environment.

These and other objects will be made manifest when considering the following detailed specification when taken in conjunction with the appended drawing figures.

### BRIEF DESCRIPTION OF THE DRAWINGS

FIG. 1 is a transparent diagrammatic representation of one embodiment of the invention packaged into a modular, plug-and-play air decontamination cartridge for use in a consumer virus eradication air purifier, or for placement in the air waste stream of industrial manufacturing facilities that emit corrosive pollutants or combustible effluents to the atmosphere.

FIG. 2 is a solid rendering of the embodiment of FIG. 1 viewed looking into the air inlet.

FIG. 3 is a solid depiction of the translucent light guide pipe dismantling rod coated with photocatalytic nanoparticles coating on the outer wall, illuminated from within the light guide via a UV energy source, also containing an inner attraction screen lining the sub-surface of the outer wall, or embedded in the outer wall.

FIG. 4 is a transparent representation of the FIG. 3 dismantling rod.

FIG. 5 is a cutaway view of the dismantling rod end showing the outer wall, photocatalytic coating applied to the outer wall surface, the inner attraction screen beneath or embedded in the outer wall, and the inner UV energy source.

FIG. 6 is an image of a tetrafluoroethylene, (trade name Teflon), pipe section (with holding tape at each end), with five (5) photocatalytic coats permanently applied to its outer surface, showing confirmation of surface free radical production by color conversion of a pattern of clear Nitroblue Tetrazolium solution that was applied to the coated pipe surface before UV illumination.

FIG. 7 is a second embodiment of the invention, packaged into a modular, plug-replaceable air decontamination cartridge with UV energy traveling down the light guide from a central source, diffusing radially outward through the coated light guide walls.

FIG. 8 is a graphical plot showing the energy emission of a deuterium lamp for purposes of measuring UV transmission and absorbance;

FIG. 9 is a graphical plot showing the deuterium lamp's emissions normalized to 100% across all wavelengths in the test range;

FIG. 10 is a graphical plot showing the transmission of an uncoated Teflon dismantling rod wall, in the UV energy range of interest;

FIG. 11 is a graphical plot showing the nearly complete absorption by the coating of 19 thin layers of photocatalytic nanoparticles applied to the outer wall surface of material such as that used in Embodiment #2.

### DETAILED DESCRIPTION OF THE INVENTION

The present invention provides a purification method and apparatus wherein contaminating particles suspended in breathable air are strongly migrated to an eradication site where they are molecularly dismantled into harmless fragments. The powerful nano-chemical agents to carry out the purification are generated on site from available natural elements, and are returned to those natural elements after their dismantling job is done, causing no environmental harm.

Dismantling Agent Creation On Site When activated by ultraviolet radiation with wavelengths shorter than 387.5 nm certain semiconductor nanoparticles produce a powerful molecular dismantling mechanism, hydroxyl radicals from ambient water molecules The hydroxyl radicals rob target molecule electrons from hydrogen atoms in the organic matter to restore their own covalent electrons lost to the energizing radiation. A colloidal suspension coating provides a stable, yet porous dry carrier structure to permanently suspend the nanoparticles, while permitting target molecules to move freely in and out of the nanoparticle matrix. The dismantling process generally applies to both gaseous chemical molecules (organic and some inorganic) and to micro-organic particles.

Invention's Optimal Use of Illuminating Radiation The present invention conformally surrounds the illuminating radiation with a dismantling surface that eradicates suspended contaminants by decomposition, thereby maximizing the use of illuminating radiation from conventionally shaped cylindrical light sources. The strength of the decontaminating process can be amplified by enhancing the migration of contaminants and ambient water vapor to the dismantling sites by electrostatic attraction.

Our research shows that ultraviolet radiation in the 250-270 nm range is very suitable for producing the photocatalytic mechanism, and for gaining the added feature of pathogen DNA/RNA destruction during the electrostatic migration step.

Internally Activated, Pathogen Migrating, Dismantling Rods This invention uses durable hollow light guides, coated with an outer layer of photocatalytic, dismantling, nanoparticles, energizing from below. These light guide dismantling rods are illuminated from within by UV radiation from an inner lamp radiating through the light guide walls to activate the photocatalytic coating from beneath. The dismantling rod purifies the contaminated air stream moving across its outer coated surface.

The light guide rod may be composed of quartz, fused silica or several other proprietary polymers (3M) that will serve for UV transmission and as the physical foundation for an active porous coating. Light guides may also be comprised of tetrafluoroethylene, (trade name Teflon). The material's extreme inertness to chemicals, low coefficient of friction, and ability to resist adhesion to almost any material, render it generally ideal for applications involving treatment of corrosive or combustible air flows. Coatings and photocatalytic activity can be on either the inside wall or the outside wall of the light guide.

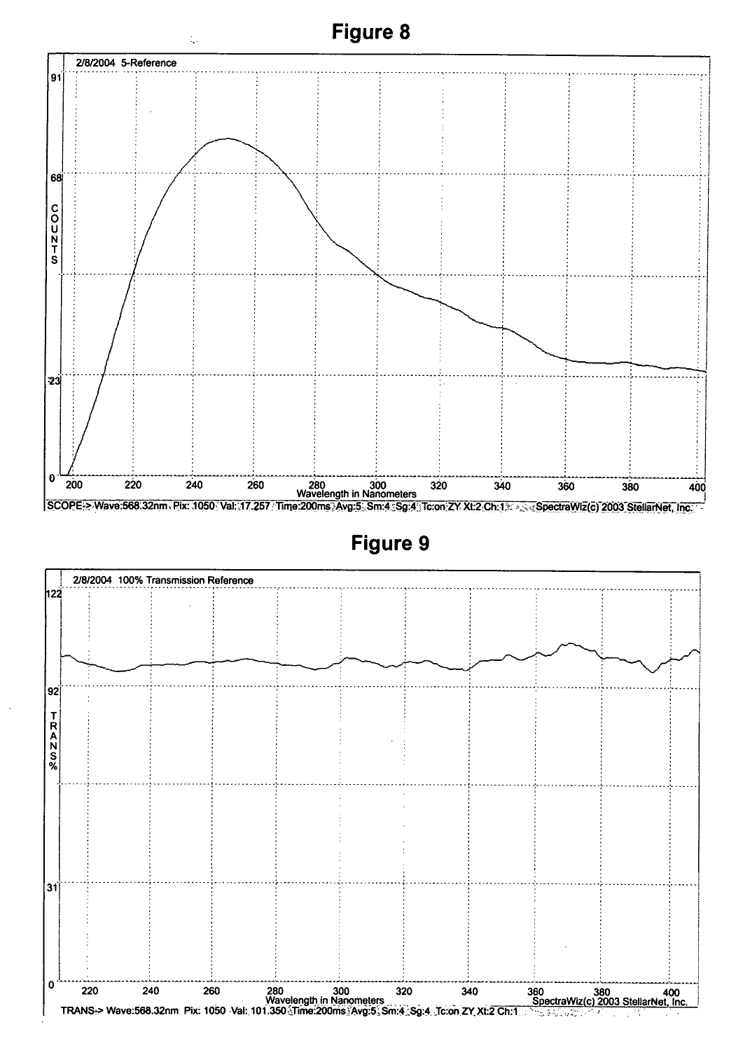

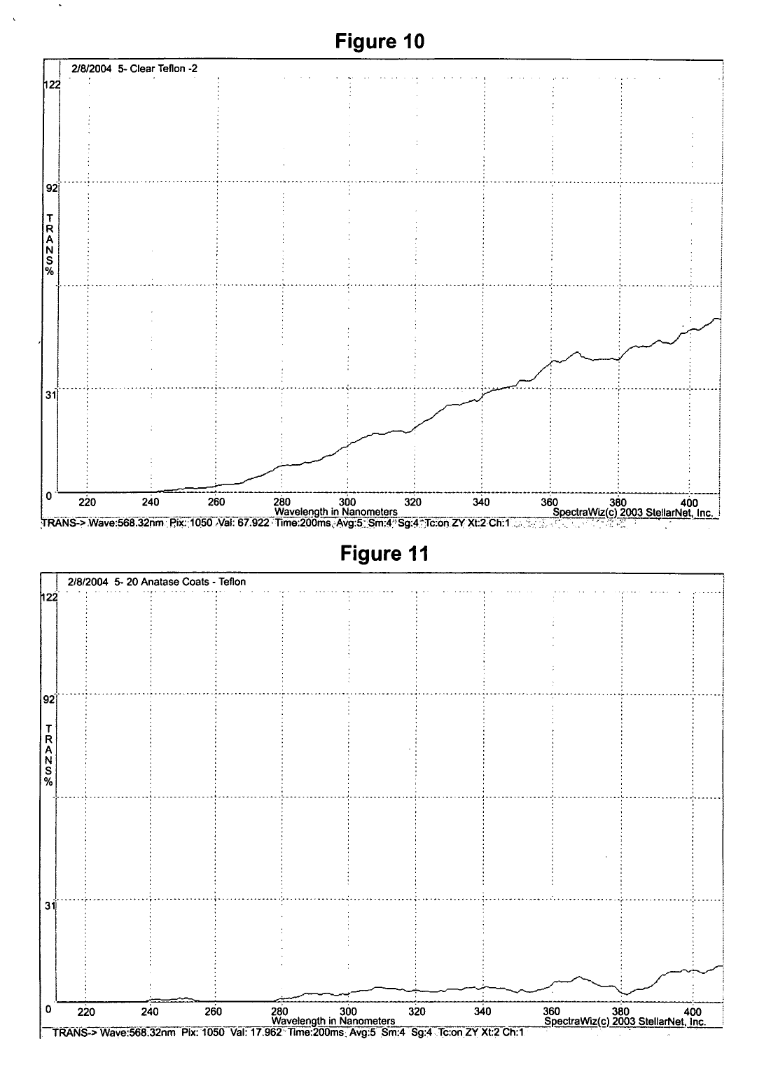

Our research shows that the latter material is an excellent transmitter of the energizing spectral range of this invention, even when modified to accept coating adhesion. FIG. 8 shows the output of a deuterium light source used to measure transmissivity of diaelectric light guide material. FIG. 9 shows the light source output normalized for 100% at every wavelength. FIG. 10 shows that our research on Teflon tubes of 0.005 inch wall thickness transmit 30% to 50% of UV radiation in the 340 nm-400 nm range. Our prototype tetrafluoroethylene tube light guides underwent molecularly permanent photocatalytic coating adcohesion, using a proprietary process not disclosed here. FIG. 11 shows that virtually all of the transmitted UV energy shown in FIG. 10 was absorbed by the adhered photocatalytic coating.

The present invention uses a process analogous to Free-Flow Electrophoeresis to help separate the contaminant particles from the moving air stream, migrating them to an activated dismantling rod. The natural negative charge of microorganisms is used and strengthened. The natural positive charge of larger particles and macromolecules is reversed to net electronegativity by charging them with electrons produced in a negative corona discharge. A negative corona is created via a negatively charged electrode in the contaminated airstream. The generation mechanism of the negative corona is different from a positive corona. With the negative electrode at high voltage potential, electrons formed by chance ionizing events gain energy from the electric field and produce ions and other electrons by collision. The positive ions are accelerated to the cathode where they bombard the electrode surface and, as a result, electrons are released from the surface. The electrons move into the weaker electric field away from the cathode where they collide with contaminant particles and chemical molecules, as well as with neutral gas molecules to form negative ions. The discharge is dependent on the surface of the electrode for electrons, so the discharge occurs in small regions, or tufts, instead of in a uniform bluish glow characteristic of a positive discharge. This negative discharge is more stable, and can achieve higher voltages and currents than a positive corona before sparkover.

The gasses N₂, CO₂, and H₂ have a much higher ionization potential than O₂ or ambient H₂O. The first three gasses tend to form positive ions in a positive corona, and (in a pure state) will not form negative ions by electron attachment. Therefore, electrons produced in a negative corona are more likely to form negative ions from O₂ and H₂O and to be available for attachment to large, positively charged contaminant particles to create their net electronegativity. This abundance of electrons in the corona is also more likely to dissociate passing O₂ molecule into its two atoms, generating higher transient ozone levels than would be created in a positive corona. Ozone production levels are dependent upon several variables, including the field strength used and the interelectrode distance. Water molecules are strongly polar with a heavy oxygen atom and two light hydrogen atoms that are constantly exchanged between water molecules. Ionization produces separate ions of H⁺ and OH⁻ which quickly form hydronium ions H₃O⁺ and hydroxyl ions OH⁻, an intermediate for hydroxyl radical •OH formation at the dismantling grid.²
² “Reactive Oxygen Species (ROS)”. Mar. 5, 2004. http://users.rcn.com/jkimball.ma.ultranet/BiologyPages/R/ROS.html.

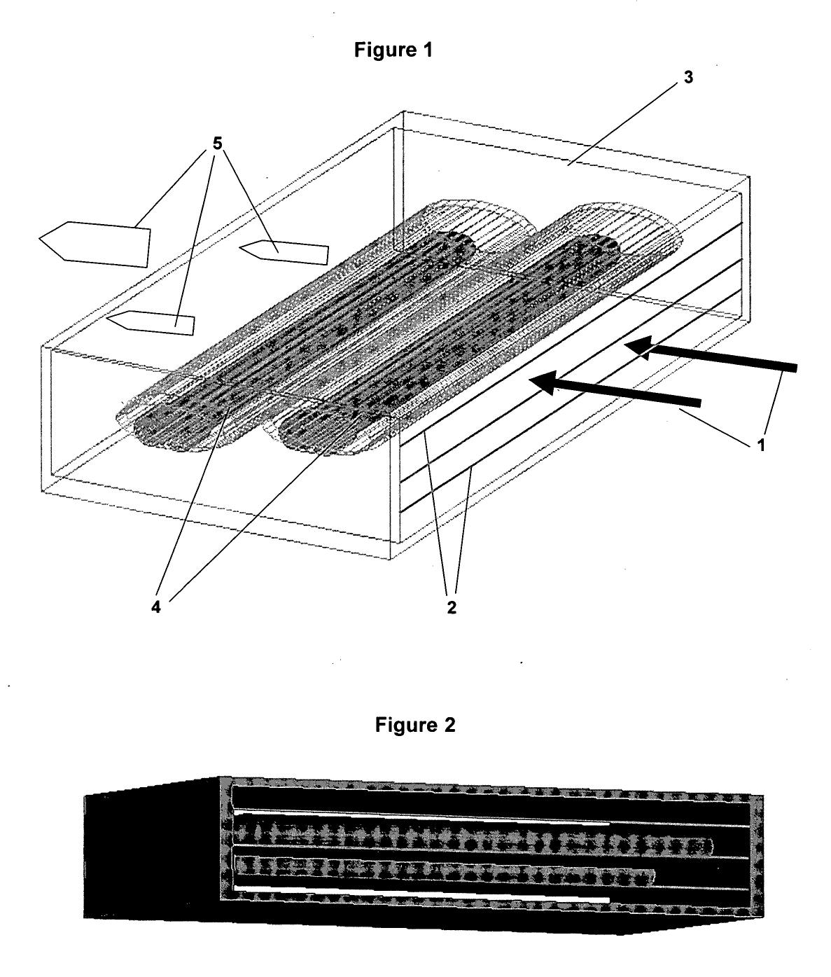

Cartridge Type Consumer Virus Eradicator FIG. 1 is a transparent diagrammatic representation of one embodiment of the invention packaged into a modular, plug-and-play air decontamination cartridge for use in a consumer virus eradication air purifier, or for placement in the air waste stream of industrial manufacturing facilities that emit corrosive pollutants or combustible effluents to the atmosphere. Contaminated air 1 is drawn into the inlet of the apparatus by a fan (not shown). Carried by the moving air, the suspended contaminants in the air pass by a negatively charged wire-based, switchable ion field generator 2 that, if switched on, increases the electronegativity of the molecule and particle contaminants' natural electrostatic surface charges. Those skilled in the art will appreciate that while a wire based ion field generator is illustrated, any mechanism for charging particles can function as the ion field generator. Carried by the moving air into the closed chamber 3, the naturally charged, or artificially charge-enhanced contaminants, and gaseous negative ions, are electrostatically migrated toward dismantling rods 4.

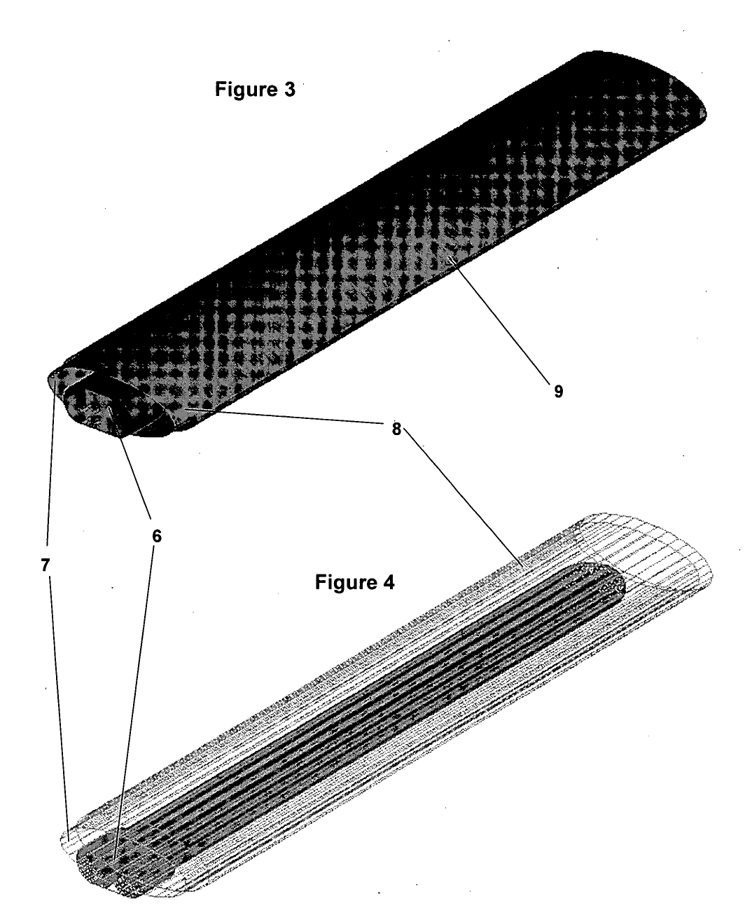

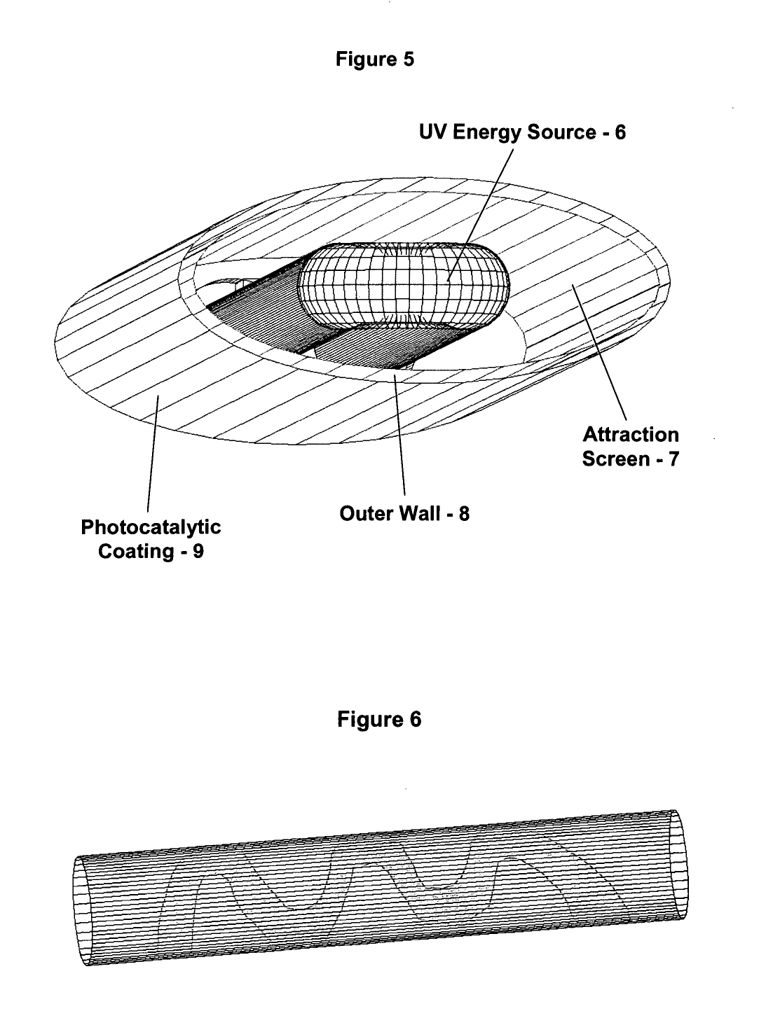

FIGS. 3, 4, and 5 show the elements of the dismantling rod 4, which includes a UV energy lamp 6, surrounded by a positively charged screen 7, that is preferably embedded in outer wall 8 or positioned internally of outer wall 8. Outer wall 8 of dismantling rod 4 dielectrically insulates the voltage and current carrying inner attracting screen 7 from the photocatalytic coating 9 on wall 8. Otherwise the surface field of dismantling free radicals on coating 9 would self-quench with charged semiconductor nanocrystal particles if the photocatalytic coating 9 were electrically charged toward either polarity.

Ambient water vapor is converted to a surface field of hydroxyl radicals by the internal UV energy source 6, whose illumination passes through both the attracting screen 7 and outer wall 8, and is absorbed by photocatalytic coating 9 on outer wall 8. FIG. 6 shows a tetrafluoroethylene, (trade name Teflon), pipe section with five (5) photocatalytic coats permanently applied to its outer surface, showing surface free radical production confirmed by color conversion of a pattern of clear Nitroblue Tetrazolium solution applied to the coated pipe surface before UV illumination.

The migrated contaminants are structurally dissociated and destroyed on the photocatalytic surface 9 of outer wall 8 where free radicals are generated. While the contaminants are being held there, the constantly replenished surface free radicals on 9 structurally dissociate the migrated, impacting contaminants into smaller and smaller fragments, eventually to natural stable molecules and harmless protein fragments. Purified air 5, (FIG. 1), exits the apparatus.

DC voltage in the lower end of the range −3000 to −30,000 volts applied at very low current to the two electrode groups is adequate for negative ionization at 2 without significant ozone production. Electrons “boil off” the wires and by diffusion collide with, or may attach to, the microorganism contaminants, further strengthening their natural net electronegativity. The larger, positively charged surfaces of inorganic particles attract and bind colliding electrons from the negative corona and negative ions created in the interelectrode space; those tend to neutralize and reverse their natural positive surface charge.

All the current in the interelectrode space is carried by the resulting negative ions. Ambient water ions, ozone ions, and charged contaminants are transported toward the oppositely charged attraction screen 7 by the influence of the electric field strength on the contaminants' natural electrostatic surface charges, or by the combined influence between 2 and 7 if the ion generation field if it is switched on. The interelectrode space is also filled with the high intensity UV field radiating through the dismantling rod's wall. Any ozone molecules present are subject to photolysis (wavelengths below 310 nm) that produces singlet oxygen, which in turn may react with a water molecule to yield more dismantling hydroxyl radicals •OH.³
³ “The Hydroxyl Radical; Sources and Measurement.” Apr. 4, 1998. http://www.chem.leeds.ac.uk/Atmospheric/Field/oh.html.

Although most of the negative ions in the particle charging field are either attached to passing contaminants or are consumed by the attracting screen's opposite polarity, those that do escape the apparatus with purified air generally freshen the escaping, breathable air conveying a beneficial psychological effect on people who inhale that air.

An alternative of this embodiment is the reversal of voltages on the ion generator wires 2 (FIG. 1) and the attracting screens 7 within the dismantling rods, either switched occasionally, or programmed to reverse automatically on a scheduled cycle. This feature helps keep the attracting rod surface clean, and can be used to enhance the device's capacity to eradicate larger particles, such as tobacco smoke, that may normally carry an inherent positive charge, rather than negative charge. The free radical surface cloud on the dismantling rod surface doesn't care whether the contaminants it encounters are negative, positive, or electroneutral—it will dismantle all of them, indiscriminately.

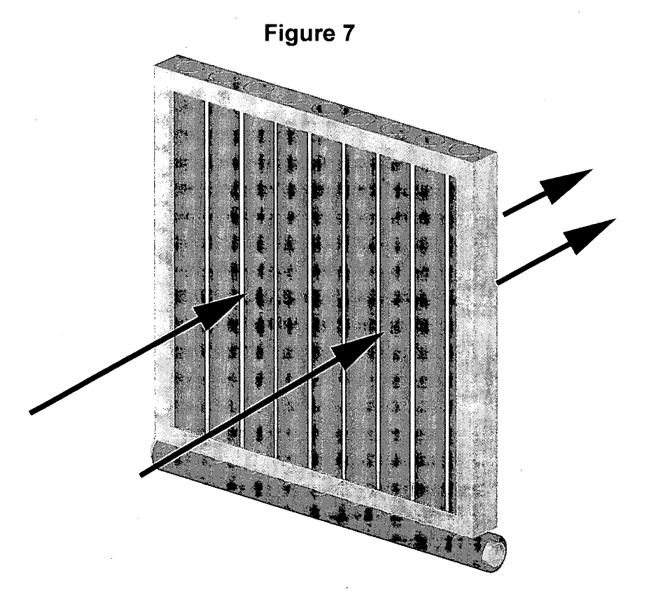

Module For Abatement and Corrosive Environments FIG. 7 shows another embodiment of the invention using an array of dismantling light guide rods illuminated from within by a central source. Contaminated airflow is purified in an in-line air handling system by passing it over an array of electrostatically attractive, photocatalytically active, coated light guide pipes made of durable diaelectric material. The light guides may be small or large diameter for either portable or high volume air flow in fixed HVAC (Heating, Ventilation, Air Conditioning) systems. UV illumination can be from a lamp within each dismantling rod, or from a single light source at one end of each light pipe array, with UV photons traveling down the tubular guide, diffusing radially outward through the coated light guide walls to activate the photocatalytic coating thereon from beneath. At the light guide end it is reflected back by a highly specular, convex reflective surface for additional, reflected diffusion into the dismantling rods. Such radiation can also be transported to the hollow dismantling rods by a flexible optical fiber bundle with fiber sub-bundles directed into each dismantling rod.

Such a cartridge is suitable for large scale placement in the air waste stream of industrial manufacturing facilities that emit corrosive pollutants or combustible effluents to the atmosphere. In our research light guides illuminated from within by such diffused longitudinal ionizing radiation, afforded amplification benefits over flat grids because of their 360 degree outer surfaces being available for contaminant attraction-dissociation.

What is claimed is:

1. An air purifier comprising:
a. a chamber having an air inlet and air outlet;
b. a dismantling rod disposed in the chamber between the air inlet and air outlet, wherein said dismantling rod comprises
c. an outer dielectric wall having an inner surface and an outer surface;
d. a current carrying material disposed inward of said inner surface;
e. a photocatalytic coating applied to the outer surface of said wall; and
f. an energy source adjacent said current carrying material.

2. A method for purifying air, said method comprising the steps of
a. directing an air stream carrying contaminants past an ion generator;
b. charging the contaminants with the ion generator;
c. attracting the charged contaminants towards a dielectric dismantling surface;
d. directing electromagnetic energy to the dismantling surface;
e. generating hydroxyl radicals on the dismantling surface utilizing said electromagnetic energy; and
f. utilizing said hydroxyl radicals to attract hydrogen molecules from said contaminant.

3. The purifier of claim 1, wherein the only chemical raw materials used to produce the dismantling agents are natural elements contained in the ambient air passing through the apparatus, including water vapor and the gaseous elements in pure air.

4. The purifier of claim 2, wherein the concentration of the purifying agents can be increased by supplementing the ambient air passing through the apparatus with additional feed gas, such as water vapor, or other feedstock that enhances production of free radicals in the apparatus.

5. The purifier of claim 1, wherein the outer layer of the dismantling rod may be constructed of tetrafluoroethylene or other durable, inorganic diaelectric material that is transmissive to the UV radiation used for photocatalytic activation.

6. The purifier of claim 1, wherein the attraction screen may be constructed of a current carrying material such as aluminum, copper, tungsten, or nickelized steel.

7. The purifier of claim 1 wherein the current carrying metal attraction screen may be embedded in the outer dismantling rod by coating the metal screen with tetrafluoroethylene or other inorganic, diaelectric material that is transmissive to the UV radiation used for photocatalytic activation.

8. The purifier of claim 2 wherein the voltage on the current carrying attraction screen is sufficient to attract the natural electrostatic surface charged contaminants entering the apparatus.

9. The purifier of claim 1, wherein the dismantling rod is coated with photocatalytic semiconductor nanoparticles, embedded in a fixed porous colloid matrix surface layer, such coating intended to create oxidizing agents that are powerful enough to dismantle the cellular and molecular structure of contaminants drawn electrostatically to the dismantling rod surface.

10. The purifier of claim 1, wherein the longitudinal placement of the energizing light source within the dismantling rod and the conformal shaping of the dismantling rod around it, permit their UV photon emission paths to radiate directly onto the maximum exposed dismantling area for maximum effectiveness on the rod surface.

11. The purifier of claim 1, wherein the UV activating plasma tubes may be driven by electrical current into electrodes embedded in the tube walls, or via electrode less tubes driven by microwave energy.

12. The purifier of claim 1, wherein the coated dismantling rod is activated by UV energy generated within the rod by energized plasma tubes, or by UV radiation emitted from a remote UV source traveling down the internal rod for radial transmission from within.

13. The purifier of claim 2, wherein contaminants suspended in the air entering the unit may be electrically excited by a strong voltage flux field that creates, or amplifies, a net electronegative charge (or a net electropositive charge) on the contaminant particles by the production of electrons, negative ions, and/or positive ions.

14. The purifier of claim 2, wherein ambient water vapor molecules suspended in the air entering the unit are also electrically excited by the ionizing field to create a net electronegative or electropositive charge on the suspended water molecules.

15. The purifier of claim 1, wherein the charged suspended contaminants, charged water vapor ions, ozone ions and other charged particles in the air stream are attracted to an oppositely charged screen or mesh which enhances the migration of the charged contaminants out of the rapidly flowing airstream, increasing the probability of their impact on the dismantling rods walls that they have been attracted to.

16. The purifier of claim 1, wherein the voltages placed on an ion field generator and the attraction screens may be reversed at time intervals, either selectively or automatically, in order to enhance capture of larger contaminant particles that may have an inherent positive charge, and to clean the dismantling surface above the attraction screen by electrically repelling any charged debris that may coat it.

17. The purifier of claim 2, wherein the activated coating structurally dissociates contaminant particles and toxic chemical compounds suspended in the airstream.

18. The purifier of claim 2, wherein the activated coating structurally dissociates any O₃ ozone produced in the apparatus back into natural O₂ oxygen molecules before the ozone molecules can escape the apparatus.

19. The purifier of claim 1, wherein use of the 240-280 nm range of UV radiation from beneath the photocatalytic coating is readily absorbed by the photocatalytic coating on the dismantling rod outer surface, and providing the further benefit of preventing further reproductive replication of passing pathogens, until they are fully eradicated, by creating unnatural peptide bonds across their deoxyribonucleic acid base pairs.

20. The purifier of claim 1, with an array of dismantling rods, that may be individually illuminated from within, or by a single lamp per rod array using light guide pipes or flexible optical fiber bundles.
